(19) United States
(12) Patent Application Publication
(10) Pub. No.: US 2025/0082775 A1
(43) Pub. Date: Mar. 13, 2025

(54) Targeted degradation and removal of amyloid beta plaques for the prevention and treatment of alzheimer’s diseases via engineered nano-scavenger exosomes

(71) Applicant: Santa Clara University, Santa Clara, CA (US)

(72) Inventors: Biao Lu, San Francisco, CA (US); Renceh Flojo, Pearl City, HI (US); Joy Ku, Sunnyvale, CA (US)

(21) Appl. No.: 18/736,217

(22) Filed: Jun. 6, 2024

Related U.S. Application Data
(60) Provisional application No. 63/471,891, filed on Jun. 8, 2023.

(51) Int. Cl. A61K 47/69; A61K 47/68

(52) U.S. Cl. CPC A61K 47/6901; A61K 47/6843; A61K 47/6847

(57) ABSTRACT

Exosome-Based Nano-Scavenger (EBNS) are provided to precisely target and remove Aβ plaques. Leveraging exosomes, natural cell-derived vesicles with tissue-penetrating capabilities, these exosomes were engineered to carry disease targeting antibodies and Aβ-degrading enzymes, clearing Aβ specifically, safely and effectively. EBNS can overcome existing treatment limitations and offer a promising avenue for Alzheimer's Disease therapy.

### CROSS-REFERENCE TO RELATED APPLICATIONS

This application claims priority from U.S. Provisional Patent Application 63/471,891 filed Jun. 8, 2023, which is incorporated herein by reference.

### FIELD OF THE INVENTION

This invention aims to produce genetically engineered nano-scavenger exosomes, capable of specifically binding and degrading amyloid beta (AB), the initiator and cause of Alzheimer's disease for the prevention and treatment of the most common type of human dementia.

### BACKGROUND OF THE INVENTION

Alzheimer's disease, the leading cause of dementia among the elderly, is characterized by the buildup of toxic Aβ proteins in the brain. Current Alzheimer's disease treatments focus on symptom management and Aβ-neutralizing therapies using recombinant human antibodies. However, in addition to their side-effects, these antibody therapies are costly, offer limited benefits in early stages, and are ineffective in late stages due to their inability to clear existing plaques and poor bio-availability.

The present invention addresses a need in the art with the development of a novel exosome-based nano-scavenger (EBNS) for the targeted removal of amyloid-beta (Aβ) proteins, offering a transformative therapeutic strategy for Alzheimer's disease (AD) and other amyloid-related disorders.

### SUMMARY OF THE INVENTION

Through genetic modification, exosomes are installed both an AβV-specific binding moiety, such as the single-chain fragment variable of antibody (scFv), and a digestive enzyme, such as the tissue plasminogen activator (tPA), urokinase plasminogen activator (UPA), insulin-degrading enzyme (IDE), or neprilysin (NEP) onto the outer surface.

These surface-engineered exosomes can be produced and subsequently purified from the conditioned medium from genetically transfected human producer 293T cells.

These surface-engineered exosomes, termed as nano-scavengers can be used as a new type of therapeutic biologics for the prevention and treatment of Alzheimer's disease.

This invention aims to establish a set of methods to produce and test the quality of engineered exosomes using a combination of assays and procedures, including differential centrifugation, ultrafiltration, chemical precipitation, confocal microscopy, nanoparticle tracking analysis, immunoassay tests, enzymatic activity analysis, cellular toxicity, and viability assay.

Nanoscale scavengers targeting extracellular disease-causing proteins have transformative impact on the potential prevention and treatment of a subset of the most-difficult-to-treat human diseases, including cancer, viral infection, and neurological disorders such as Alzheimer's disease (AD). AD causes a progressive loss of memory and cognitive abilities and ultimately a total loss of neurological function and death. As such, the typical life expectancy of AD following diagnosis is 8˜10 years. The current standard of treatment is systemic management and palliative. Most recently, Aduhelm (aducanumab) is the only monoclonal antibody directed against amyloid beta approved by the FDA for the treatment of AD. Although this potential disease-modifying antibody therapy slows cognitive decline of AD by 27%, its marginal benefit is associated with adverse effects, which have contributed to the deaths of at least two trial participants among other safety concerns. This disease imposes a huge financial burden on both patients and society, with an estimated global annual cost of 1 trillion US dollars.

Exosomes are human cell-derived nanoscale extracellular vesicles that can carry large amounts of bioactive cargo such as proteins, enzymes, nucleic acids and lipids, and shuttle them among various tissues and organs. Owning to the intrinsic ability of high tissue penetration, exosomes can reach the highly insulated brain tissues. Therefore nano-scavenger exosomes represent a promising strategy for the better treatment of brain diseases such as AD. This invention aims to develop an engineering strategy to produce genetically engineered exosomes, namely the nano-scavenger, capable of specifically binding to Aβ: first to prevent their aggregation to form toxic Aβ-plaques, and secondly to degrade them by co-displayed enzymes via several mechanisms (FIG. 1).

This invention demonstrates a genetic approach for the production nano-scavengers by displaying these two critical functional moieties on the surface of exosomes. This type of nano-scavenger will provide a better solution to the prevention and treatment of Alzheimer's disease with enhanced potency, safety and precision due to its dual-ability of specific binding and degradation.

### A Novel and Complete Nano-Platform

Nanomedicine holds great promise to enhance safety, potency and precision of the next generation of medicine. Among various nanotechnologies, the recently discovered human cell-derived nanovesicles, termed exosomes, exhibit outstanding properties that are superior over other nanomaterials including liposomes, polymers, and virus-like particles. These advantages include 100% biocompatibility, bioavailability, biodegradability, concentrated drug-loading, and an intrinsic accessibility to the brain tissues. In this invention, we will combine three independent technologies, namely antibody engineering, genetic fusion, and exosome surface display to create an exosome-based nano-platform, enabling a streamlined production of surface-functionalized exosomes for the targeted degradation and clearance of Aβ for the prevention and treatment of Alzheimer's disease.

The three key technologies are:
(1) Design and generation of scFv via antibody engineering and reverse genetics. A molecular protocol is provided for identifying, retrieving, mapping, clustering, and linking of antigen binding sequences of monoclonal antibodies that are capable of binding to human Aβ. This molecular protocol will be employed to assemble single or dual scFv modules, which are subsequently combined with a surface display scaffold and displayed onto the outer surface of exosomes in living human producer cells (FIGS. 2A-C).
(2) Design and build of a collection of endogenous enzymes that are capable of enzymatically digesting amyloid beta. A collection of endogenous enzymes based is selected and functionally tested on their degrading ability, structure and membrane topology. We will select four types of candidate enzymes for system validation. We will use synthetic biology to generate the enzyme gene sequences and then fuse them with an exosome-targeting scaffolds for the surface display (FIGS. 3A-B).
(3) Develop a set of streamlined protocols to produce exosome-based nano-scavenger via surface display technology using cultured human 293T cells (FIG. 4).
   Design and Construction of scFv Via Antibody Engineering and Reverse Genetics

Single-chain variable fragment (scFv) is generated by linking the variable heavy chain (VH) and the variable light chain (VL) of any Aβ antibody. Compared with the 4-chain monoclonal antibody, scFv is a short and single chain equivalent, which can be fused to an exosome-targeting scaffold for surface display. Successful construction of the scFv relies on the length and sequence of VH and VL, as well as the length and flexibility of linker peptides. Optimal length and sequences of these sequences (VH, VL, and linker peptides) are implemented according to the conserved domain structure of VH and VL. The amino-acids for linkers are based on amino-acid repeats of GGGGS, a common linker frequently used for scFv construction. Back-translation tool is used to generate nucleic acid sequences which the specified amino-acid sequences have originated from. This online software takes in the amino-acid sequences as input and is able to generate corresponding coding sequences according to the preferred codon table chosen by the user. It provides a genetic tool to streamline the design of expression vectors for scFv and other chimeric genes for nano-scavenger production.

### Exosome as Novel Nanocarrier

The field of exosome-based biologics has expanded over the past decade. Reports on loading of various cargos such as chemotherapeutics, siRNA, mRNA and proteins have firmly established that these human cell-derived exosomes can be used in nanomedicine. Compared to existing therapeutic biologics, exosomes exhibit unique particle size (50˜150 nm), a high surface area to volume ratio that offers scFv and enzyme presentation. Through immuno-binding and enzymatic digestion, the Aβ monomer, oligomer, or plaque can be effectively destroyed via molecular interactions. In case of binding but not fully digested via presented enzymes, these Aβ captured exosomes can be endocytosed and degraded by surveillance cells such as macrophage or microglia in the brain.

Genetic Loading of Two Functional Moieties onto the Surface of Exosomes

Embodiments of the invention provide a strategy to install two therapeutic proteins on the outer surface of exosomes in a living human cultured cell. Using a genetic method to produce exosome-based therapeutics is novel and has a number of advantages over the existing monoclonal antibody therapies, which have limited efficacy but significant side effects. Compared to the soluble monoclonal antibody therapy, engineered nano-scavenger exosomes are more potent and efficient, safer, and more precise. For instance, each engineered exosome can carry many therapeutic molecules with an intrinsic ability to penetrate deeper into brain tissues. Plus, they can prevent both Aβ plaque formation and removal via either specific binding or degradation, thus more potent than antibody therapy. Furthermore, nano-scavenger exosomes can be up-taken via a receptor-mediated endocytosis, a highly effective mechanism for intracellular degradation of exosome-bound Aβ.—Together, these unique intrinsic features of nano-scavenger exosomes may guarantee a highly effective mechanism for intracellular degradation of exosome-bound Aβ, and subsequently clearance of neuron-toxic waste.

### Novel Brain-Deliverable Anti-Aβ Nanomedicine

By exploiting the unique properties of exosomes, one could then produce both scFv and human enzyme-displayed exosomes for the targeted clearance of Aβ accumulated in Alzheimer's brain. The human cell-derived exosomes possess an intrinsic ability to cross the brain-blood-barrier (BBB), targeted to the major disease-cause agent Aβ. Specific removal of extracellular toxic Aβ waste represents a novel class of drug targets implicated in several human diseases such as Alzheimer's and other amyloid-accumulated diseases associated with metabolic disorders and cancers.

### A Modular Design

The unique features of the genetic design of embodiments of this invention for producing engineered exosomes are multi-faceted. First, it enables loading of disease-targeting moiety, i.e. scFv, at the outer surface. Displaying at the outside rather than inside will allow for more functional moieties installed, enhancing the potency of exosome therapy. Because scFv and most of the extracellular enzymes used lack the transmembrane sequences, we will provide and include an exosome-anchoring scaffold into our design. This molecular scaffold contains three motifs to ensure the proper display of functional moiety and they are: 1) the flexible linker; 2) a transmembrane helix; and 3) intraluminal end. Second, by tagging fluorescence-emitting proteins such as green/red fluorescence proteins, these chimeric proteins can be monitored in live cells and in real-time via confocal microscopy, thus providing a mechanism for quality control of scFv expression, processing and incorporation into engineered exosomes. Together, embodiments of this invention will be valuable for planning preclinical studies (FIGS. 2A-C, 3A-B, and 4).

As stated infra the nano-scavenger platform of this invention includes three key components:
(1) Design and generation of scFv via antibody engineering and reverse genetics;
(2) Design and build of a collection of endogenous enzymes that are capable of enzymatically digest Aβ;
(3) Develop a set of streamlined protocols to produce exosome-based nano-scavengers via surface display technology using cultured human 293T cells. The built-in molecular monitoring system will enable real-time monitoring of molecular incorporation of displayed protein cargo for quality control of the production and purification process.

Because of the modular structure of the molecular design, it is possible to test each construct individually. After successful validation of each construct, the simultaneous incorporation of two or more functional moieties, such as scFv and enzymes, into engineered exosome can be easily achieved by co-transfections.

### Variations and Modifications

Dual-functional nano-scavengers for Aβ clearance could arise from the success of this invention. Due to the modular nature, it's also possible to use the same targeting and degrading strategy for other diseases, as a lot of them are caused by aberrant protein misfolding and aggregation. For examples, scavenger exosomes can also be used for the treatment of thrombotic disorders such as myocardial infarction (MI) and stroke, as well as other amyloidosis including light chain, serum amyloid A, dialysis-related Aβ2M and hereditary and old age wild-type transferrin amyloid, etc.

The present invention provides in one embodiment a genetically engineered nano-scavenger exosome capable of binding and degrading a protein. The genetically engineered nano-scavenger exosome has an expression vector with a protein-specific binding moiety. The protein-specific binding moiety is selected from the group consisting of a Single-Chain Fragment Variable (scFv), a Tissue Plasminogen Activator (tPA), Urokinase (UPA), an Insulin-Degrading eEnzyme (IDE), and a Neprilysin (NEP). The protein-specific binding moiety is attached to an exosome, where the protein-specific binding moiety is capable of binding and degrading the protein.

In this embodiment, the protein-specific binding moiety is selected from the group consisting of SEQ. ID. No.1, SEQ ID. No 2 and SEQ ID No. 3.

In this embodiment, the expression vector further has a signal peptide, where the signal peptide is SEQ. ID. No. 4, SEQ. ID. No. 5, SEQ. ID. No. 6, SEQ. ID. No. 7, SEQ. ID. No. 8 and SEQ. ID. No. 9.

In this embodiment, the expression vector further has a hinge sequence such as SEQ. ID. No. 10.

In this embodiment, the expression vector further has a VSVG-transmembrane helix such as SEQ. ID. No. 11.

In this embodiment, the expression vector further has an GFP reporter protein such as SEQ. ID. No. 12.

The protein can be an amyloid beta (Aβ), a Tau protein, an alpha-synuclein, a huntingtin protein, a prion protein, an amyloid light chain (AL) protein, an amyloid A (AA) protein, a transthretin (TTR), an atrial natriuretic peptide (ANP), an islet amyloid polypeptide (IAPP), a gelsolin, an apolipprotein a-I, an apolopoprotein A-II, a cystatin C, a fibrinogen A alpha-chain, a prolactin, a calcitonin, a keratoepithelin or a blood clot.

Embodiments of the invention involve developing a novel nano-medicine, called Exosome-Based Nano-Scavenger (EBNS), to precisely target and remove Aβ plaques. Leveraging exosomes, natural cell-derived vesicles with tissue-penetrating capabilities, the inventors engineered these exosomes to carry disease targeting antibodies and Aβ-degrading enzymes, clearing Aβ specifically, safely and effectively. EBNS can overcome existing treatment limitations and offer a promising avenue for Alzheimer's Disease therapy. This scavenger platform can be easily adopted to treat other amyloid-related disorders.

### BRIEF DESCRIPTION OF THE DRAWINGS

For color interpretation of the drawings, the reader is referred to priority document U.S. Provisional Patent Application 63/471,891 filed Jun. 8, 2023.

FIG. 1 shows according to an exemplary embodiment of the invention a schematic illustration of mechanisms of scavenger exosomes for targeted binding and removal of beta amyloid in human brain. The targeted clearance of amyloid beta plaques by scavenger exosomes through four potential mechanisms: (1) binding and disgusting of newly released beta amyloid monomers to prevent oligomerization. (2) binding and digesting oligomers to prevent them from formation of toxic plaques. (3) binding and on-site digestion of plaques. (4) binding and removal of plaque residues via exosome uptake and intracellular recycling in microglia and astrocytes in brain. The black Y symbol on exosome surface indicates antibody binding domain toward beta amyloid. The red pie symbol on exosome surface indicates digestive enzymes of amyloid beta.

FIGS. 2A-C show according to an exemplary embodiment of the invention genetic engineering of exosomes for targeted removal of amyloid beta. FIG. 2A shows the configuration of expression vectors for targeted Aβ removal. FIG. 2B shows the modular and functional domain of genetic modifiers. FIG. 2C shows the membrane topology of scFv and enzymes.

FIGS. 3A-B show according to an exemplary embodiment of the invention genetic engineering of exosomes via molecular targeting. FIG. 3A shows the biogenesis of exosomes and microvesicles in human cells are illustrated. FIG. 3B shows the molecular pathways of exosome-targeting and enzyme-loading strategy are depicted.

FIG. 4 shows according to an exemplary embodiment of the invention production and characterization of exosome-based nano-scavengers.

FIG. 5 shows according to an exemplary embodiment of the invention potential cutting sites at amyloid beta (Aβ) by various proteolytic enzymes. The most abundant peptides are Aβ (1-40) and Aβ (1-42) with a ratio of roughly 10:1. Aβ (1-42) is significantly more neurotoxic and is also the major constituent of senile plaques. Black arrows: NEP cutting sites; ‘Blue’ (B) arrows: IDE cutting sites; and ‘Red’ arrows: tPA/UPA cutting sites.

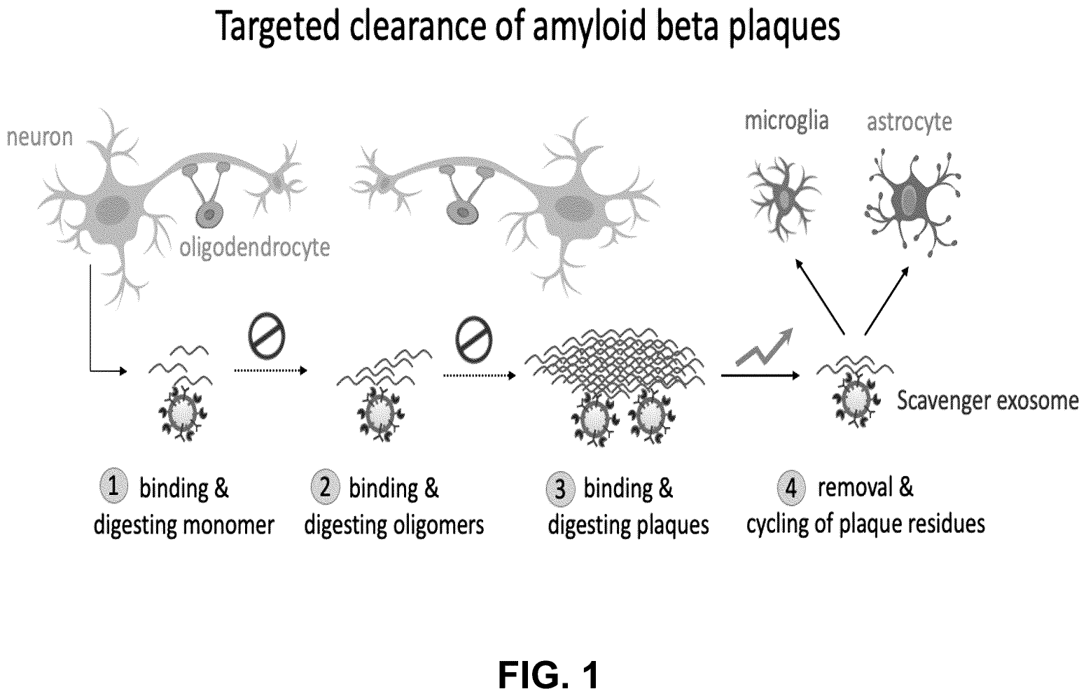

### DETAILED DESCRIPTION

Selection, Design, and Exosome Surface Display of a Collection of Endogenous Enzymes that are Capable of Enzymatically Digesting Aβ Plaques

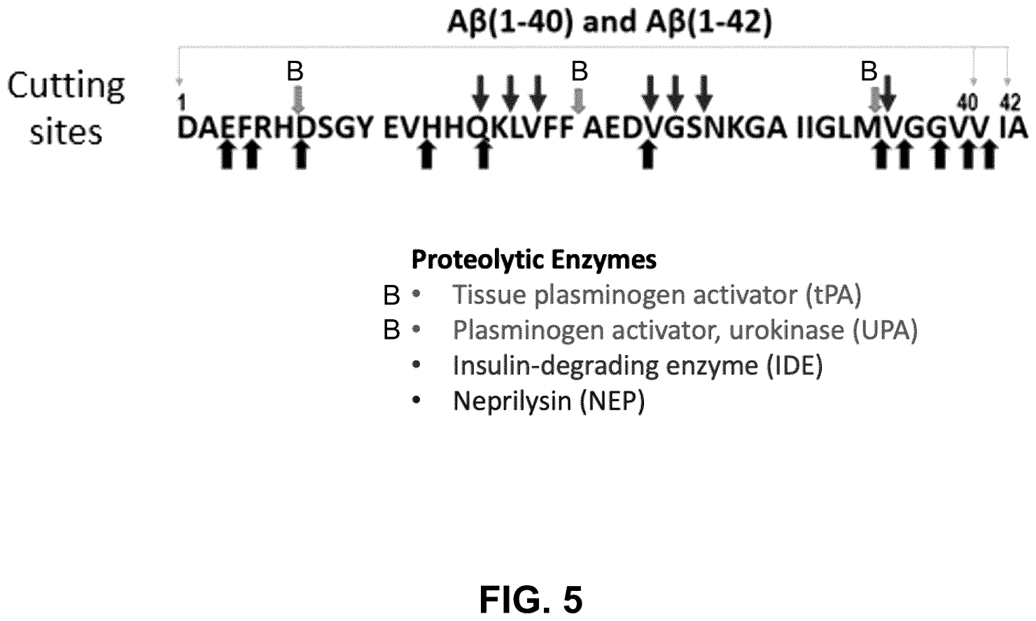

Selection of proteolytic enzymes: a protease also termed as a peptidase, protease or proteolytic enzyme, is an enzyme that catalyzes the break down proteins into smaller polypeptides or single amino acids. Proteolytic enzymes may function intracellularly or extracellularly. Based on catalytic residue, they can be classified into several groups including serine-, cysteine-, aspartic-, and metallo-proteases. These can be used for the purpose of degrading extracellular protein garbage Aβ to treat human Alzheimer's, we formulate the following criteria for choosing the suitable protease candidates. They include: 1) have 2 or more potential cutting sites on Aβ; 2) optimally work in extracellular environments, namely natural extracellular proteases; 3) coenzyme and factors are either not required or readily available in the extracellular environments; 4) these proteases are of human origin; and 5) they are manageable for the surface display via a genetic approach such as exosome-surface display technology. According to these criteria, we have identified four protease enzymes from UniProt (http://www.uniprot.org/), which contains comprehensive and freely accessible resources of protein sequence and functional information (Table 1). FIG. 5 illustrates the four protease candidates and their potential cutting sites on Aβ.

TABLE 1
Sequence IDs of enzyme, antibody, scaffold and reporter

| | UniProt.No., |
|---|---|
| | DrugBank.No, |
| | or other |
| Enzyme/antibody/domain | database IDs |
| Tissue-type plasminogen activator (tPA) | P00750-1 |
| Plasminogen activator, Urokinase (uPA) | P00749 |
| Insulin-degrading enzyme (IDE) | P14735-1 |
| Neprilysin (NEP) | P08473 |
| Aducanumab | D10541 |
| Lecanemab | D11678 |
| Signal peptide-Mammalia | ID7209 |
| Hinge from CD8 alpha chain | P01732-1 |
| VSVG transmembrane domain (tVSVG) | VSVGP03522 |
| Enhanced green fluorescence protein | A0A076V611 |
| (EGFP) | |

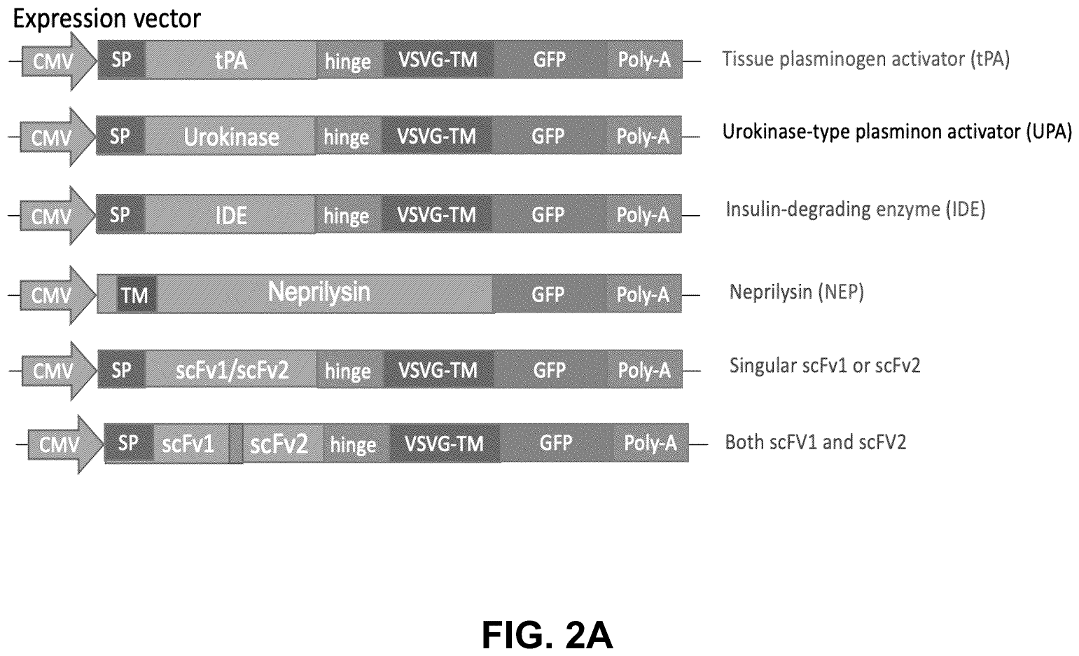

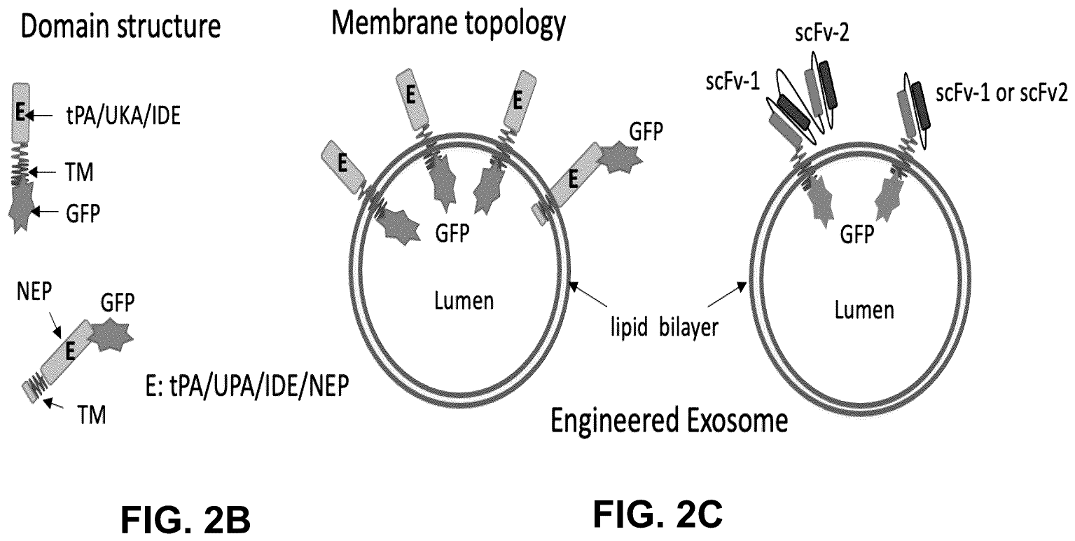

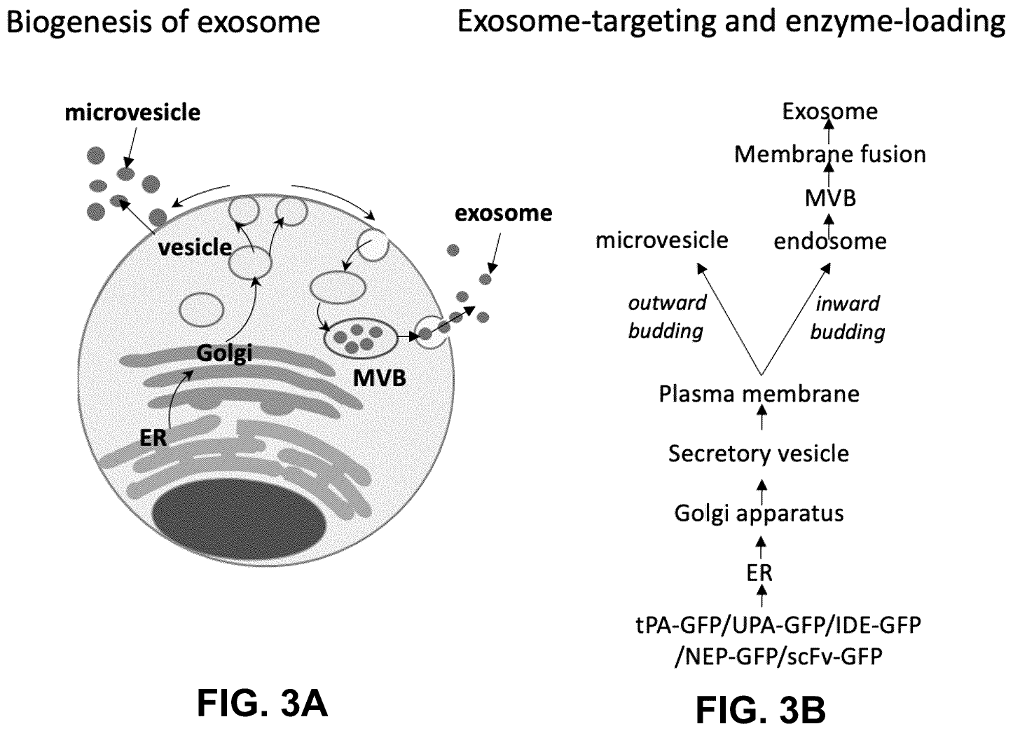

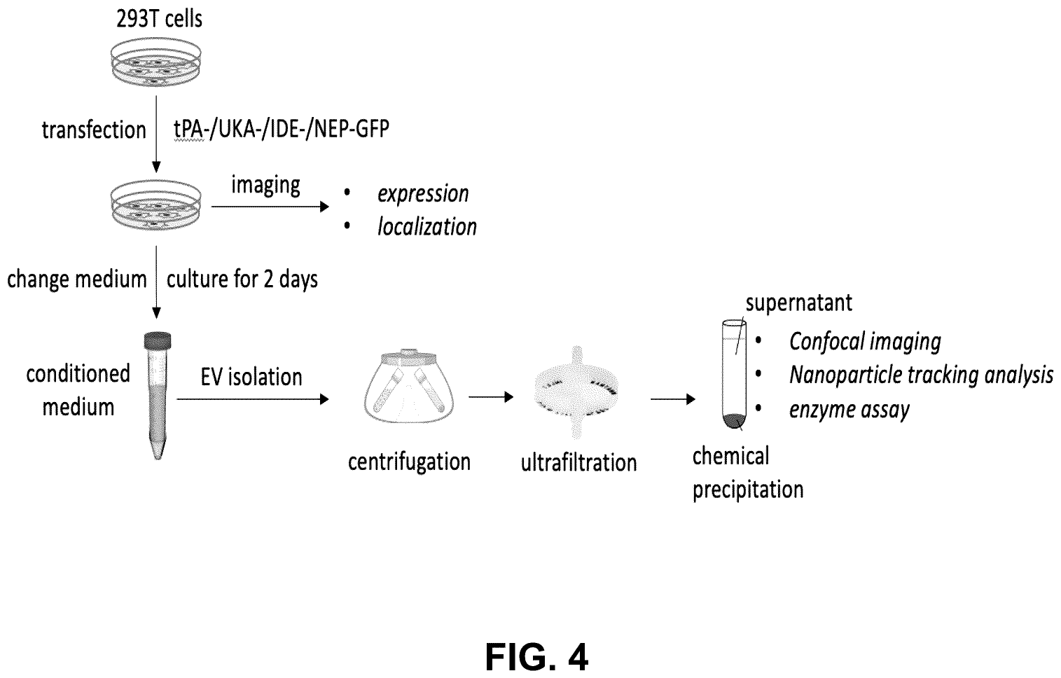

Design and exosome surface display strategy: Domain analysis of candidate enzymes indicates that tissue plasminogen activator (tPA), urokinase (UPA), and insulin-degrading enzyme (IDE) are secreted as extracellular enzymes, with each possessing a signal peptide at its N-terminus. Thus, C-terminal fusion with exosome targeting and anchoring scaffolds will provide a mechanism to display these enzymes at the outer surface of exosomes. According to this design, the final genetic construct will include the following sequences: 1) a signal peptide; 2) proteolytic enzyme; 3) transmembrane scaffold for exosome membrane targeting and anchoring; and 4) a shorter intraluminal end, which can in turn be tagged by monitoring reporters such as GFP (FIG. 2A). Human neprilysin (NEP), a single-pass transmembrane protein, has an extracellular C-terminus and cytoplasmic N-terminus. Therefore, NEP does not need an exosome-targeting scaffold for exosome membrane anchoring. For the purpose of molecular monitoring, a GFP reporter is tagged to the C-terminus (FIGS. 2A-C). Transfection of cultured 293T cells with the aforementioned construct will result in expression and display of NEP-GFP on the outer surface of the exosome (FIG. 2C).

### Construction of Mammalian Expression Vectors Via DNA Synthesis:

Expression is constructed by synthesis and insertion of the coding sequences for scFv and chimeric enzymes in pcDNA3.1 (Thermo Fisher Scientific) via a gene synthesis service provided by Genscript (Genscript Biotech Corp.). Specifically, the DNA sequences of scFv and exosome-targeting scaffold are synthesized by synthetic biology using a DNA synthesis service at Genscript. The synthesized DNA is subsequently subcloned into HindIII and EcoRV sites under the promoter of CMV and followed by a polyadenylation signal (Poly A). FIG. 2A illustrates the configuration of each chimeric construct used for expression of engineered exosomes harboring either scFv or proteolytic enzymes on their surface.

Design and Assembly of scFv Via Antibody Engineering and Reverse Genetics

The inventors have developed an experimental protocol that can identify and retrieve all essential amino-acid sequences from the candidate monoclonal antibodies to assemble scFv. Table 1 summarizes the monoclonal antibody and their DrugBank accession No. for the retrieval of antibody sequences. The scFv is derived from variable regions of the heavy chain (VH) and light chain (VL) of these monoclonal antibodies. The VH fragment is the N-terminal sequences of ˜108 amino acids (aa), while the VL fragment is the N-terminal sequences of ˜104 aa. To form a single fragment of variables (scFv), we join the two fragments (VL and VH) with a 20-aa linker (KRTGGGGSGGGGSGGGGSEV, based on a common generic GGGGS linker sequence, as defined supra). Within the linker, the underlined amino-acids indicate three and two aa sequences flanking the VL and VH respectively. To ensure proper display at the outer surface of exosomes, a conservative signal peptide (MLLLVTSLLLCELPHPAFLLIPD, Signal Peptide Database-Mammalia, ID7209) is tagged at the N-terminus of the scFv, while an exosome-targeted scaffold is added at the C-terminus. According to this design, the final genetic construct will include the following sequences: 1) a signal peptide; 2) scFv; 3) transmembrane scaffold for exosome membrane targeting and anchoring; and 4) a shorter intraluminal end, which can be in turn be tagged by monitoring reporters such as GFP (FIG. 2A-C).

Configuration and sequence information of chimeric enzymes and sc Fv used for exosome surface display: Molecular configuration of chimeric enzymes used for the production of scavenger exosomes are documented as example infra. The full-length enzyme of tPA, UPA, or IDE enzymes is fused with hinge sequences of the human CD8, then transmembrane helix and its adjacent sequences of VSVG and followed by enhanced GFP (EGFP) coding sequences. For NEP, since these enzymes contain its own transmembrane domain, so the full-length NEP is tagged with EGFP via a flexible linker of “GGGSGGGGSAS” (a two-time repeats based on a common generic GGGGS linker sequence, as defined supra). The scFv derived from either monoclonal antibody Aducanumb, Lecanemab or both Aducanumb and Lecanemab are tagged with additional polypeptides including a signal peptide of “MLLLVTSLLLCELPHPAFLLIP” (Signal Peptide Database-Mammalia, ID7209) at the N-terminus and a linker (GGGS, based on a common generic GGGGS linker sequence, as defined supra) followed by the human CD8 hinge, VSVG-transmembrane helix, and EGFP at the C-terminus. The amino-acid sequences are provided and annotated infra.

### Development of a Set of Streamlined Protocols to Produce Exosome-Based Nano-Scavengers Via Surface Display Technology Using Cultured Human 293T Cells

The following sections summarize materials and methods used for engineered exosome production, exosome preparation and characterization.

Materials and reagents: Human kidney cells (293T) were purchased from Alsterm (Richmond, CA). High glucose DMEM, Opti-MEM medium, and fetal bovine serum were purchased from Thermo Fisher Scientific (Waltham, MA). Chemical defined and serum-free (UltraCulture medium) was purchased from Lonza (Prtsmouth, NH). Transfection reagent polyethylenimine (PEI) was from Sigma-Aldrich (St. Louis, MO). Lipofectamine2000 from Invitrogen, and FuGene6 from Promega (Madison, WI). Nuclear staining solution Hoechst 33342 was purchased from ThermoFisher Scientific (Fremont, CA). Exosome precipitation solution (ExoQuick-TC) was obtained from System Biosciences (Palo Alto, CA). 30 mL BD syringes and 0.2 μm syringe filters were obtained from VWR International (Radnor, PA).

Expression vector construction, plasmid DNA preparation and sequence confirmation: All expression vectors were constructed using a commercial DNA synthesis and subcloning service from Genscript. Briefly, the coding sequences of scFv and proteolytic enzymes and the exosome-targeting scaffold are generated by synthetic biology and cloned in frame with the GFP reporter under the control of cytomegalovirus promoter (CMV). All final constructs were verified by double stranded DNA sequencing to ensure fidelity.

Cell culture and transfection: Human 293T cells were maintained in high-glucose DMEM (Invitrogen) supplemented with 10% fetal bovine serum (Invitrogen), 2 mM GlutMax and 100 U/mL (Gibco), 2 mM GlutaMax and 100 U/mL penicillin-streptomycin (Gibco). Cell cultures were incubated at 37° C. and equilibrated in 5% CO₂ and air. For transient transfection, cells were grown to ˜40-50% confluency before transfection by addition of plasmid DNA (1˜2 μg/well) mixed with either Lipofectamine 2000 (Invitrogen), FuGene6 (Promega), or PEI (Sigma-Aldrich) transfection reagents. Transfected cells were cultured and monitored for up to 3˜4 days for short-term observations.

Fluorescence and confocal microscopy: Images of cultured live cells or fluorescently labeled exosomes were recorded using Leica TCS SP8 confocal microscope (See priority document FIGS. 6-7) To demonstrate intracellular localization of the genetically produced nano-scavengers, both fluorescence and light transmitted images from the same filed were recorded and merged (See priority document FIGS. 7-8). Image adjustments such as brightness and contrast were applied to the entire image frame using instrument software.

Production of engineered exosomes: To prepare modified exosomes, a 150 mm tissue culture plate was used for each sample with 15 mL of DMEM (Gibco Dulbecco's Modified Eagle Medium)+10% FBS+1% Pen Strep. 350 ul of cells was then added to each plate and were incubated until reaching 70-80% confluency after ˜3 days. Subsequently, each plate was transfected by 2 ml preparation containing 20 μg of DNA, 100 μL of PEI, each diluted in 1 mL of OPTIMEM. The combined DNA and PEI was allowed to rest for 20 minutes before adding into the 150 mm plate. Cells were then incubated for additional 24 hours, followed by replacement with 15 mL of UltraCulture or Opti-MEM media to allow exosome releasing and accumulation in the conditioned medium for additional 48 hours.

Preparation of engineered exosomes from conditioned medium: Exosomes were prepared and purified from the conditioned medium with a combination of centrifugation, ultrafiltration and chemical precipitation. Human kidney 293T cells seeded on 150 mm culture plates were grown to 60˜70% confluency. For each plate, 20 μg plasmid was mixed with PEI and added to the culture medium to allow transfection to occur for 24 hr. Then the transfection media was replaced with Ultraculture to allow accumulation of exosomes in media for an additional 48 hours. The conditioned medium was subjected to centrifugation for 10 min at 1500 g to remove large cell debris. The resulting supernatant was then filtered through 0.2 μm filters. Finally, the filtered media was mixed with ExoQuick-TC and incubated at 4° C. overnight. Subsequently, the samples were centrifuged at 3000 g for 1.5 hours to pellet exosomes. The resulting pellet (exosomes) was re-suspended in PBS and stored at −20° C. or −80° C. for future use.

Confocal imaging of the engineered exosomes: For each exosome sample, 3˜10 μL of exosomes in PBS were added onto a 35 mm imaging plate to be imaged with the confocal microscope. In priority document FIG. 9 the inventors showed the successfully engineered exosomes (GFP positive), while native exosomes are GFP negative.

Nanoparticle tracking analysis: Nanoparticle tracking analysis was conducted using a NanoSight LM10 instrument with a 405 nm and 60 mV laser sources. Typically, 1 mL of a diluted exosome sample is subjected to laser light scattering and Brownian motion of particles were recorded and analyzed for sizing. Size distribution of exosomes were calculated and graphed by the NTA software. NTA was used to characterize the particle concentration, size and size distribution of each preparation. NTA analysis of exosomes demonstrates that a single peak of vesicle population with a mode size ranging from 70˜110 nm, similar to those of non-modified controls (See priority document FIG. 10).

Validation of the targeted nano-scavenger system: The successful engineering of nano-scavenger exosomes can be tested by detection of surface displayed therapeutic enzymes and binding scFv. Specifically, Proteolytic enzymes such as tPA, UPA, IDE, and NEP can be quantified using enzyme linked immunosorbent assay (ELISA) or enzymatic activity assay. Neprilysin activity was determined using a fluorometric activity assay kit (Category Number. MAK350 from Sigma Aldrich). The NEP-transfected and unmodified exosomes were diluted to 1 mg/ml proteins and 5 μl per sample were assayed by following manual instructions. The concentrations of NEP enzymes (ng/mL) were calculated according to the standard cure and recombinant NEP controls (See priority document FIG. 11).

### Sequences

SEQ. ID. No.1
D10541 (Aducanumab) with Sequence:
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PLTFGGGTKVEIKRTGGGGSGGGGSGGGGSEVQLVESGGGVVQPGRS
LRLSCAASGFAFSSYGMHWVRQAPGKGLEWVAVIWEDGTKKYYTDSV
KGRFTISRDNSKNTLYLQMNTLRAEDTAVYYCARDRGIGARRGPYYM
DVWGKGTTVTVSSAST
SEQ. ID. No.2
D11678 (Lecanemab) with Sequence:
VVMTQSPLSLPVTPGAPASISCRSSQSIVHSNGNTYLEWYLQKPGQS
PKLLIYKVSNRFSGVPDRFSGSGSGTDFTLRISRVEAEDVGIYYCFQ
GSHVPPTFGPGTKLEIKRTGGGGSGGGGSGGGGSEVQLVESGGGLVQ
PGGSLRLSCSASGFTFSSFGMHWVRQAPGKGLEWVAYISSGSSTIYY
GDTVKGRFTISRDNAKNSLFLQMSSLRAEDTAVYYCAREGGYYYGRS
YYTMDYWGQGTTVTVSSAST
SEQ. ID. No. 3
Both D10541 (Aducanumab) and D11678 (Lecanemab)
with Sequence:
DIQMTQSPSSLSASVGDRVTITCRASQSISSYLNWYQQKPGKAPKLL
IYAASSLQSGVPSRFSGSGSGTDETLTISSLQPEDFATYYCQQSYST
PLTFGGGTKVEIKRTGGGGSGGGGSGGGGSEVQLVESGGGVVQPGRS
LRLSCAASGFAFSSYGMHWVRQAPGKGLEWVAVIWEDGTKKYYTDSV
KGRFTISRDNSKNTLYLQMNTLRAEDTAVYYCARDRGIGARRGPYYM
DVWGKGTTVTVSSASTKGPSVFPLAPSSSGSGEVQLVESGGGLVQPG
GSLRLSCSASGFTFSSFGMHWVRQAPGKGLEWVAYISSGSSTIYYGD
TVKGRFTISRDNAKNSLFLQMSSLRAEDTAVYYCAREGGYYYGRSYY
TMDYWGQGTTVTVSSGGGGSGGGGSGGGGSVVMTQSPLSLPVTPGAP
ASISCRSSQSIVHSNGNTYLEWYLQKPGQSPKLLIYKVSNRFSGVPD
RFSGSGSGTDFTLRISRVEAEDVGIYYCFQGSHVPPTFGPGTKLEIK
RTVAAPSVFIFPPSA

SEQ. ID. No. 4 through SEQ. ID. No. 9 as follows are partial sequences of each listed gene, namely the signal peptide. Because these enzymes have their own signal peptide, we do not choose to add additional peptide sequences. Therefore, SEQ. ID No. 4 through SEQ. ID. No. 9 are partial sequences, but added here for completion.

SEQ. ID. No. 4
P00750-1(tPA) with Sequence:
MDAMKRGLCCVLLLCGAVEVSPSQEIHAR
SEQ. ID. No. 5
P00749(uPA) with Sequence:
MRALLARLLLCVLVVSDSKGS
SEQ. ID. No. 6
P14735-1(IDE) with Sequence:
MRYRLAWLLHPALPSTFRSVLGA
SEQ. ID. No. 7
D10541(Aducanumab) with Sequence:
MLLLVTSLLLCELPHPAFLLIP
SEQ. ID. No. 8
D11678(Lecanemab) with Sequence:
MLLLVTSLLLCELPHPAFLLIP
SEQ. ID. No. 9
D10541 and D11678 with Sequence:
MLLLVTSLLLCELPHPAFLLIP
SEQ. ID. No. 10
ID7209(signal peptide database-Mammalia) with
sequence:
MLLLVTSLLLCELPHPAFLLIP
SEQ. ID. No. 11
P01732-1 (human CD8 alpha chain) with Sequence:
TTTPAPRPPTPAPTIASQPLSLRPEACRPAAGGAVHTRGLDFACD
SEQ. ID. No. 12
VSVGP03522(VSVG) with Sequence:
EHPHIQDAASQLPDDESLFFGDTGLSKNPIELVEGWESSWKSSIASF
FFIIGLIIGLFLVLRVGIHLCIKLKHTKKRQIYTDIEMNRLGK
SEQ. ID. No. 13
A0A076V611 (EGFP) with Sequence:
MSKGEELFTGVVPILVELDGDVNGHKFSVSGEGEGDATYGKLTLKFI
CTTGKLPVPWPTLVTTLTYGVQCESRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK
SEQ. ID. No. 14
P00750-1(tPA) with Sequence:
MDAMKRGLCCVLLLCGAVFVSPSQEIHARFRRGARSYQVICRDEKTQ
MIYQQHQSWLRPVLRSNRVEYCWCNSGRAQCHSVPVKSCSEPRCFNG
GTCQQALYFSDFVCQCPEGFAGKCCEIDTRATCYEDQGISYRGTWST
AESGAECTNWNSSALAQKPYSGRRPDAIRLGLGNHNYCRNPDRDSKP
WCYVEKAGKYSSEFCSTPACSEGNSDCYFGNGSAYRGTHSLTESGAS
CLPWNSMILIGKVYTAQNPSAQALGLGKHNYCRNPDGDAKPWCHVLK
NRRLTWEYCDVPSCSTCGLRQYSQPQFRIKGGLFADIASHPWQAAIF
AKHRRSPGERFLCGGILISSCWILSAAHCFQERFPPHHLTVILGRTY
RVVPGEEEQKFEVEKYIVHKEFDDDTYDNDIALLQLKSDSSRCAQES
SVVRTVCLPPADLQLPDWTECELSGYGKHEALSPFYSERLKEAHVRL
YPSSRCTSQHLLNRTVTDNMLCAGDTRSGGPQANLHDACQGDSGGPL
VCLNDGRMTLVGIISWGLGCGQKDVPGVYTKVTNYLDWIRDNMRP
SEQ. ID. No. 15
P00749(uPA) with Sequence:
MRALLARLLLCVLVVSDSKGSNELHQVPSNCDCLNGGTCVSNKYFSN
IHWCNCPKKFGGQHCEIDKSKTCYEGNGHFYRGKASTDTMGRPCLPW
NSATVLQQTYHAHRSDALQLGLGKHNYCRNPDNRRRPWCYVQVGLKL
LVQECMVHDCADGKKPSSPPEELKFQCGQKTLRPRFKIIGGEFTTIE
NQPWFAAIYRRHRGGSVTYVCGGSLISPCWVISATHCFIDYPKKEDY
IVYLGRSRLNSNTQGEMKFEVENLILHKDYSADTLAHHNDIALLKIR
SKEGRCAQPSRTIQTICLPSMYNDPQFGTSCEITGFGKENSTDYLYP
EQLKMTVVKLISHRECQQPHYYGSEVTTKMLCAADPQWKTDSCQGDS
GGPLVCSLQGRMTLTGIVSWGRGCALKDKPGVYTRVSHFLPWIRSHT
KEENGLAL
SEQ. ID. No. 16
P08473 (NEP) with Sequence:
MGKSESQMDITDINTPKPKKKQRWTPLEISLSVLVLLLTIIAVTMIA
LYATYDDGICKSSDCIKSAARLIQNMDATTEPCTDFFKYACGGWLKR
NVIPETSSRYGNFDILRDELEVVLKDVLQEPKTEDIVAVQKAKALYR
SCINESAIDSRGGEPLLKLLPDIYGWPVATENWEQKYGASWTAEKAI
AQLNSKYGKKVLINLFVGTDDKNSVNHVIHIDQPRLGLPSRDYYECT
GIYKEACTAYVDFMISVARLIRQEERLPIDENQLALEMNKVMELEKE
IANATAKPEDRNDPMLLYNKMTLAQIQNNESLEINGKPESWLNFTNE
IMSTVNISITNEEDVVVYAPEYLTKLKPILTKYSARDLQNLMSWRFI
MDLVSSLSRTYKESRNAFRKALYGTTSETATWRRCANYVNGNMENAV
GRLYVEAAFAGESKHVVEDLIAQIREVFIQTLDDLTWMDAETKKRAE
EKALAIKERIGYPDDIVSNDNKLNNEYLELNYKEDEYFENIIQNLKF
SQSKQLKKLREKVDKDEWISGAAVVNAFYSSGRNQIVFPAGILQPPF
FSAQQSNSLNYGGIGMVIGHEITHGFDDNGRNFNKDGDLVDWWTQQS
ASNFKEQSQCMVYQYGNFSWDLAGGQHLNGINTLGENIADNGGLGQA
YRAYQNYIKKNGEEKLLPGLDLNHKQLFFLNFAQVWCGTYRPEYAVN
SIKTDVHSPGNFRIIGTLQNSAEFSEAFHCRKNSYMNPEKKCRVW
SEQ. ID. No. 17
P14735-1(IDE) with Sequence:
MRYRLAWLLHPALPSTFRSVLGARLPPPERLCGFQKKTYSKMNNPAI
KRIGNHITKSPEDKREYRGLELANGIKVLLISDPDRFAQFFLCPLFD
ESCKDREVNAVDSEHEKNVMNDAWRLFQLEKATGNPKHPFSKFGTGN
KYTLETRPNQEGIDVRQELLKFHSAYYSSNLMAVCVLGRESLDDLTN
LVVKLFSEVENKNVPLPEFPEHPFQEEHLKQLYKIVPIKDIRNLYVT
FPIPDLQKYYKSNPGHYLGHLIGHEGPGSLLSELKSKGWVNTLVGGQ
KEGARGFMFFIINVDLTEEGLLHVEDIILHMFQYIQKLRAEGPQEWV
FQECKDLNAVAFRFKDKERPRGYTSKIAGILHYYPLEEVLTAEYLLE
EFRPDLIEMVLDKLRPENVRVAIVSKSFEGKTDRTEEWYGTQYKQEA
IPDEVIKKWQNADLNGKFKLPTKNEFIPTNFEILPLEKEATPYPALI
KDTAMSKLWFKQDDKFFLPKACLNFEFFSPFAYVDPLHCNMAYLYLE
LLKDSLNEYAYAAELAGLSYDLQNTIYGMYLSVKGYNDKQPILLKKI
IEKMATFEIDEKRFEIIKEAYMRSLNNFRAEQPHQHAMYYLRLLMTE
VAWTKDELKEALDDVTLPRLKAFIPQLLSRLHIEALLHGNITKQAAL
GIMQMVEDTLIEHAHTKPLLPSQLVRYREVQLPDRGWFVYQQRNEVH
NNCGIEIYYQTDMQSTSENMFLELFCQIISEPCFNTLRTKEQLGYIV
FSGPRRANGIQGLRFIIQSEKPPHYLESRVEAFLITMEKSIEDMTEE
AFQKHIQALAIRRLDKPKKLSAECAKYWGEIISQQYNFDRDNTEVAY
LKTLTKEDIIKFYKEMLAVDAPRRHKVSVHVLAREMDSCPVVGEFPC
QNDINLSQAPALPQPEVIQNMTEFKRGLPLFPLVKPHINEMAAKL

### Examples

Reader is also referred to Appendix B in the priority document.

>tPA-tVSVG-EGFP-935aa

This sequence is built as follows with the indication of the respective SEQ. ID. Nos.

(SEQ. ID. No. 14 tPA)
MDAMKRGLCCVLLLCGAVFVSPSQEIHARFRRGARSYQVICRDEKTQ
MIYQQHQSWLRPVLRSNRVEYCWCNSGRAQCHSVPVKSCSEPRCFNG
GTCQQALYFSDFVCQCPEGFAGKCCEIDTRATCYEDQGISYRGTWST
AESGAECTNWNSSALAQKPYSGRRPDAIRLGLGNHNYCRNPDRDSKP
WCYVFKAGKYSSEFCSTPACSEGNSDCYFGNGSAYRGTHSLTESGAS
CLPWNSMILIGKVYTAQNPSAQALGLGKHNYCRNPDGDAKPWCHVLK
NRRLTWEYCDVPSCSTCGLRQYSQPQFRIKGGLFADIASHPWQAAIF
AKHRRSPGERFLCGGILISSCWILSAAHCFQERFPPHHLTVILGRTY
RVVPGEEEQKFEVEKYIVHKEFDDDTYDNDIALLQLKSDSSRCAQES
SVVRTVCLPPADLQLPDWTECELSGYGKHEALSPFYSERLKEAHVRL
YPSSRCTSQHLLNRTVTDNMLCAGDTRSGGPQANLHDACQGDSGGPL
VCLNDGRMTLVGIISWGLGCGQKDVPGVYTKVTNYLDWIRDNMRP
(SEQ. ID. No. 10 human CD8 alpha chain)
TTTPAPRPPTPAPTIASQPLSLRPEACRPAAGGAVHTRGLDFACD
(SEQ. ID. No. 12 tVSVG)
EHPHIQDAASQLPDDESLFFGDTGLSKNPIELVEGWFSSWKSSIASF
FFIIGLIIGLFLVLRVGIHLCIKLKHTKKRQIYTDIEMNRLGK
(SEQ. ID. No. 13 EGFP)
MSKGEELFTGVVPILVELDGDVNGHKFSVSGEGEGDATYGKLTLKFI
CTTGKLPVPWPTLVTTLTYGVQCESRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK*

(SEQ. ID. No. 15. uPA)
MRALLARLLLCVLVVSDSKGSNELHQVPSNCDCLNGGTCVSNKYFSN
IHWCNCPKKFGGQHCEIDKSKTCYEGNGHFYRGKASTDTMGRPCLPW
NSATVLQQTYHAHRSDALQLGLGKHNYCRNPDNRRRPWCYVQVGLKL
LVQECMVHDCADGKKPSSPPEELKFQCGQKTLRPRFKIIGGEFTTIE
NQPWFAAIYRRHRGGSVTYVCGGSLISPCWVISATHCFIDYPKKEDY
IVYLGRSRLNSNTQGEMKFEVENLILHKDYSADTLAHHNDIALLKIR
SKEGRCAQPSRTIQTICLPSMYNDPQFGTSCEITGFGKENSTDYLYP
EQLKMTVVKLISHRECQQPHYYGSEVTTKMLCAADPQWKTDSCQGDS
GGPLVCSLQGRMTLTGIVSWGRGCALKDKPGVYTRVSHELPWIRSHT
KEENGLAL
(SEQ. ID. No. 11 human CD8 alpha chain)
TTTPAPRPPTPAPTIASQPLSLRPEACRPAAGGAVHTRGLDFACD
(SEQ. ID. No. 12 tVSVG)
EHPHIQDAASQLPDDESLFFGDTGLSKNPIELVEGWFSSWKSSIASE
FFIIGLIIGLFLVLRVGIHLCIKLKHTKKRQIYTDIEMNRLGK
(SEQ. ID. No. 13 EGFP)
MSKGEELFTGVVPILVELDGDVNGHKFSVSGEGEGDATYGKLTLKFI
CTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK*

(SEQ. ID. No. 16 NEP)
MGKSESQMDITDINTPKPKKKQRWTPLEISLSVLVLLLTIIAVTMIA
LYATYDDGICKSSDCIKSAARLIQNMDATTEPCTDFFKYACGGWLKR
NVIPETSSRYGNFDILRDELEVVLKDVLQEPKTEDIVAVQKAKALYR
SCINESAIDSRGGEPLLKLLPDIYGWPVATENWEQKYGASWTAEKAI
AQLNSKYGKKVLINLFVGTDDKNSVNHVIHIDQPRLGLPSRDYYECT
GIYKEACTAYVDEMISVARLIRQEERLPIDENQLALEMNKVMELEKE
IANATAKPEDRNDPMLLYNKMTLAQIQNNFSLEINGKPESWLNETNE
IMSTVNISITNEEDVVVYAPEYLTKLKPILTKYSARDLQNLMSWRFI
MDLVSSLSRTYKESRNAFRKALYGTTSETATWRRCANYVNGNMENAV
GRLYVEAAFAGESKHVVEDLIAQIREVFIQTLDDLTWMDAETKKRAE
EKALAIKERIGYPDDIVSNDNKLNNEYLELNYKEDEYFENIIQNLKF
SQSKQLKKLREKVDKDEWISGAAVVNAFYSSGRNQIVFPAGILQPPF
FSAQQSNSLNYGGIGMVIGHEITHGFDDNGRNFNKDGDLVDWWTQQS
ASNFKEQSQCMVYQYGNFSWDLAGGQHLNGINTLGENIADNGGLGQA
YRAYQNYIKKNGEEKLLPGLDLNHKQLFFLNFAQVWCGTYRPEYAVN
SIKTDVHSPGNFRIIGTLQNSAEFSEAFHCRKNSYMNPEKKCRVW
(SEQ. ID. No. 13 EGFP)
GGGGSGGGGSAS (linker)
MSKGEELFTGVVPILVELDGDVNGHKFSVSGEGEGDATYGKLTLKFI
CTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK*

(SEQ. ID. No. 17 IDE)
MRYRLAWLLHPALPSTERSVLGARLPPPERLCGFQKKTYSKMNNPAI
KRIGNHITKSPEDKREYRGLELANGIKVLLISDPTTDKSSAALDVHI
GSLSDPPNIAGLSHFCEHMLFLGTKKYPKENEYSQFLSEHAGSSNAF
TSGEHTNYYFDVSHEHLEGALDRFAQFFLCPLFDESCKDREVNAVDS
EHEKNVMNDAWRLFQLEKATGNPKHPFSKFGTGNKYTLETRPNQEGI
DVRQELLKFHSAYYSSNLMAVCVLGRESLDDLTNLVVKLFSEVENKN
VPLPEFPEHPFQEEHLKQLYKIVPIKDIRNLYVTFPIPDLQKYYKSN
PGHYLGHLIGHEGPGSLLSELKSKGWVNTLVGGQKEGARGEMFFIIN
VDLTEEGLLHVEDIILHMFQYIQKLRAEGPQEWVFQECKDLNAVAFR
FKDKERPRGYTSKIAGILHYYPLEEVLTAEYLLEEFRPDLIEMVLDK
LRPENVRVAIVSKSFEGKTDRTEEWYGTQYKQEAIPDEVIKKWQNAD
LNGKFKLPTKNEFIPTNFEILPLEKEATPYPALIKDTAMSKLWFKQD
DKFFLPKACLNFEFFSPFAYVDPLHCNMAYLYLELLKDSLNEYAYAA
ELAGLSYDLQNTIYGMYLSVKGYNDKQPILLKKIIEKMATFEIDEKR
FEIIKEAYMRSLNNFRAEQPHQHAMYYLRLLMTEVAWTKDELKEALD
DVTLPRLKAFIPQLLSRLHIEALLHGNITKQAALGIMQMVEDTLIEH
AHTKPLLPSQLVRYREVQLPDRGWFVYQQRNEVHNNCGIEIYYQTDM
QSTSENMFLELFCQIISEPCENTLRTKEQLGYIVESGPRRANGIQGL
RFIIQSEKPPHYLESRVEAFLITMEKSIEDMTEEAFQKHIQALAIRR
LDKPKKLSAECAKYWGEIISQQYNEDRDNTEVAYLKTLTKEDIIKFY
KEMLAVDAPRRHKVSVHVLAREMDSCPVVGEFPCQNDINLSQAPALP
QPEVIQNMTEFKRGLPLFPLVKPHINEMAAKL
(SEQ. ID. No. 11 human CD8 alpha chain)
TTTPAPRPPTPAPTIASQPLSLRPEACRPAAGGAVHTRGLDFACD
(SEQ. ID. No. 12 tVSVG)
EHPHIQDAASQLPDDESLFFGDTGLSKNPIELVEGWESSWKSSIASE
FFIIGLIIGLFLVLRVGIHLCIKLKHTKKRQIYTDIEMNRLGK
(SEQ. ID. No. 13 EGFP)
MSKGEELFTGVVPILVELDGDVNGHKFSVSGEGEGDATYGKLTLKFI
CTTGKLPVPWPTLVTTLTYGVQCESRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNEKIRHNIEDGSVQLADHYQQNTPI
GDGPVLLPDNHYLSTQSALSKDPNEKRDHMVLLEFVTAAGITHGMDE
LYK*

(SEQ. ID. No. 9 Signal peptide)
MLLLVTSLLLCELPHPAFLLIP
(SEQ. ID. No. 1. Aducanumab)
DIQMTQSPSSLSASVGDRVTITCRASQSISSYLNWYQQKPGKAPKLL
IYAASSLQSGVPSRESGSGSGTDETLTISSLQPEDFATYYCQQSYST
PLTFGGGTKVEIKRTGGGGSGGGGSGGGGSEVQLVESGGGVVQPGRS
LRLSCAASGFAFSSYGMHWVRQAPGKGLEWVAVIWFDGTKKYYTDSV
KGRFTISRDNSKNTLYLQMNTLRAEDTAVYYCARDRGIGARRGPYYM
DVWGKGTTVTVSSAST
(S EQ. ID. No. 11 human CD8 alpha chain)
GGGGS(linker)
TTTPAPRPPTPAPTIASQPLSLRPEACRPAAGGAVHTRGLDFACD
(SEQ. ID. No. 12 tVSVG)
EHPHIQDAASQLPDDESLFFGDTGLSKNPIELVEGWESSWKSSIASF
FFIIGLIIGLFLVLRVGIHLCIKLKHTKKRQIYTDIEMNRLGKDIQH
SGGR
(SEQ. ID. No. 13 EGFP)
MSKGEELFTGVVPILVELDGDVNGHKFSVSGEGEGDATYGKLTLKFI
CTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK*

(SEQ. ID. No. 8 Signal peptide)
MLLLVTSLLLCELPHPAFLLIP
(SEQ. ID. No. 2. Lecanemab)
VVMTQSPLSLPVTPGAPASISCRSSQSIVHSNGNTYLEWYLQKPGQS
PKLLIYKVSNRFSGVPDRESGSGSGTDETLRISRVEAEDVGIYYCFQ
GSHVPPTFGPGTKLEIKRTGGGGSGGGGSGGGGSEVQLVESGGGLVQ
PGGSLRLSCSASGFTFSSFGMHWVRQAPGKGLEWVAYISSGSSTIYY
GDTVKGRFTISRDNAKNSLFLQMSSLRAEDTAVYYCAREGGYYYGRS
YYTMDYWGQGTTVTVSSAST
(SEQ. ID. No. 11 human CD8 alpha chain)
GGGGS (linker)
TTTPAPRPPTPAPTIASQPLSLRPEACRPAAGGAVHTRGLDFACD
(SEQ. ID. No. 12 tVSVG)
EHPHIQDAASQLPDDESLFFGDTGLSKNPIELVEGWESSWKSSIASF
FFIIGLIIGLFLVLRVGIHLCIKLKHTKKRQIYTDIEMNRLGKDIQH
SGGR
(SEQ. ID. No. 13 EGFP)
MSKGEELFTGVVPILVELDGDVNGHKFSVSGEGEGDATYGKLTLKFI
CTTGKLPVPWPTLVTTLTYGVQCESRYPDHMKQHDFFKSAMPEGYVQ
ERTIFFKDDGNYKTRAEVKFEGDTLVNRIELKGIDFKEDGNILGHKL
EYNYNSHNVYIMADKQKNGIKVNFKIRHNIEDGSVQLADHYQQNTPI
GDGPVLLPDNHYLSTQSALSKDPNEKRDHMVLLEFVTAAGITHGMDE
LYK*

What is claimed is:

1. A genetically engineered nano-scavenger exosome capable of binding and degrading a protein, the genetically engineered nano-scavenger exosome comprising an expression vector with a protein-specific binding moiety, wherein the protein-specific binding moiety is selected from the group consisting of a Single-Chain Fragment Variable (scFv), a Tissue Plasminogen Activator (tPA), Urokinase (UPA), an Insulin-Degrading eEnzyme (IDE), and a Neprilysin (NEP), wherein the protein-specific binding moiety is attached to an exosome, and wherein the protein-specific binding moiety is capable of binding and degrading the protein.

2. The genetically engineered nano-scavenger exosome as set forth in claim 1, wherein the protein-specific binding moiety is selected from the group consisting of SEQ. ID. No.1, SEQ ID. No 2 and SEQ ID No. 3.

3. The genetically engineered nano-scavenger exosome as set forth in claim 1, wherein the expression vector further comprises a signal peptide, wherein the signal peptide is SEQ. ID. No. 4, SEQ. ID. No. 5, SEQ. ID. No. 6, SEQ. ID. No. 7, SEQ. ID. No. 8 and SEQ. ID. No. 9.

4. The genetically engineered nano-scavenger exosome as set forth in claim 1, wherein the expression vector further comprises a hinge sequence, wherein the hinge sequence is SEQ. ID. No. 10.

5. The genetically engineered nano-scavenger exosome as set forth in claim 1, wherein the expression vector further comprises a VSVG-transmembrane helix, wherein the VSVG-transmembrane helix is SEQ. ID. No. 11.

6. The genetically engineered nano-scavenger exosome as set forth in claim 1, wherein the expression vector further comprises an GFP reporter protein, wherein the GFP reporter protein is SEQ. ID. No. 12.

7. The genetically engineered nano-scavenger exosome as set forth in claim 1, wherein the protein is an amyloid beta (Aβ), a Tau protein, an alpha-synuclein, a huntingtin protein, a prion protein, an amyloid light chain (AL) protein, an amyloid A (AA) protein, a transthretin (TTR), an atrial natriuretic peptide (ANP), an islet amyloid polypeptide (IAPP), a gelsolin, an apolipprotein a-I, an apolopoprotein A-II, a cystatin C, a fibrinogen A alpha-chain, a prolactin, a calcitonin, a keratoepithelin or a blood clot.


SEQUENCE LISTING (17 sequences)

SEQ ID NO: 1 - protein, 251 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSAST

SEQ ID NO: 2 - protein, 255 aa, unidentified
VVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSAST

SEQ ID NO: 3 - protein, 532 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTGGGGSGGGGSGGGGSEVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSSGSGEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSGGGGSGGGGSGGGGSVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSA

SEQ ID NO: 4 - protein, 29 aa, unidentified
MDAMKRGLCCVLLLCGAVFVSPSQEIHAR

SEQ ID NO: 5 - protein, 21 aa, unidentified
MRALLARLLLCVLVVSDSKGS

SEQ ID NO: 6 - protein, 23 aa, unidentified
MRYRLAWLLHPALPSTFRSVLGA

SEQ ID NO: 7 - protein, 22 aa, unidentified
MLLLVTSLLLCELPHPAFLLIP

SEQ ID NO: 8 - protein, 22 aa, unidentified
MLLLVTSLLLCELPHPAFLLIP

SEQ ID NO: 9 - protein, 22 aa, unidentified
MLLLVTSLLLCELPHPAFLLIP

SEQ ID NO: 10 - protein, 22 aa, unidentified
MLLLVTSLLLCELPHPAFLLIP

SEQ ID NO: 11 - protein, 45 aa, unidentified
TTTPAPRPPTPAPTIASQPLSLRPEACRPAAGGAVHTRGLDFACD

SEQ ID NO: 12 - protein, 90 aa, unidentified
EHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 13 - protein, 238 aa, unidentified
MSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 14 - protein, 562 aa, unidentified
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 15 - protein, 431 aa, unidentified
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKLLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 16 - protein, 750 aa, unidentified
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 17 - protein, 938 aa, unidentified
MRYRLAWLLHPALPSTFRSVLGARLPPPERLCGFQKKTYSKMNNPAIKRIGNHITKSPEDKREYRGLELANGIKVLLISDPDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL